(19) United States
(12) Patent Application Publication
(10) Pub. No.: US 2007/0037174 A1
(43) Pub. Date: Feb. 15, 2007

(54) Chemiluminescent generated fluorescent labeling

(76) Inventors: Jang B. Rampal, Yorba Linda, CA (US); Robert S. Matson, Orange, CA (US)

Correspondence Address: BECKMAN COULTER INC.;C/O SHELDON MAK ROSE & ANDERSON, 225 SOUTH LAKE AVENUE, 9TH FLOOR, PASADENA, CA, 91101, US

(21) Appl. No.: 11/203,073

(22) Filed: Aug. 12, 2005

(51) Int. Cl. C12Q 1/68; G01N 33/53

(52) U.S. Cl. 435/6; 435/7.5

(57) ABSTRACT

A method for detecting a target analyte in a sample having the steps of: binding the target analyte to a solid substrate; binding a reporter to the bound target to form a label enzyme; contacting a chemiluminescent reagent with the label enzyme to produce a fluorescent precipitate; and detecting fluorescence from the fluorescent precipitate.

### BACKGROUND

The present invention relates generally to biomolecular assays, and more particularly, to a system and method for detecting target analytes in a sample.

Reactions between biological molecules exhibit an extremely high degree of specificity, which provides a living cell with the ability to carry out thousands of chemical reactions simultaneously in the same vessel. Generally, this specificity arises from the fit between two molecules having very complex surface topologies. An example of this is an antibody binding to a molecule displaying an antigen on its surface, because the antibody contains a pocket whose shape is the complement of a protruding area on an antigen. Tests that detect the presence of DNA or RNA that is complementary to a known DNA or RNA chain are based upon the sequences in the chains such that an A in one chain is always matched to a T in the other chain, and a C in one chain is always matched to a G in the other chain, binding the two chains together by electrostatic forces.

Systems for medical diagnosis often involve a bank of tests, each test measuring the binding of one mobile component to a corresponding immobilized component. To provide inexpensive test kits, systems having a matrix of immobilized spots have been suggested wherein each spot includes the immobilized component of a two component test such as described above. The fluid to be tested is typically brought into contact with the matrix. After rinsing away unbound sample, the presence of the analyte within the sample is determined by its location within the matrix using labeled markers to determine the presence of the analyte.

However, there is a need for improved systems and methods for detecting analytes.

### SUMMARY

Accordingly, the present invention is directed to a method for detecting a target analyte in a sample comprising the steps of: binding the target analyte to a solid substrate; binding a reporter to the bound target to form a label enzyme; contacting a chemiluminescent reagent with the label enzyme to produce a fluorescent precipitate; and detecting fluorescence from the fluorescent precipitate.

The contacting of the chemiluminescent reagent with the label enzyme may also produce chemiluminescence. The produced chemiluminescence may be used to quantify the amount of target bound. The contacting of the chemilumincscent reagent with the label enzyme may also produce a visible precipitate observable to the naked eye or imaged using a camera system.

Optionally, the solid substrate is at least one of the group consisting of glass, organic thin film, inorganic thin film, polymer, self-assembled monolayers, and a microparticle. Optionally, the solid substrate is at least one of the group consisting of foams, filaments, threads, sheets, films, slides, gels, membranes, tapes, compact disks, microplates and beads. Optionally, the solid substrate is a plate having discrete isolated areas in the form of at least one of the group consisting of wells, troughs, pedestals, hydrophobic patches, hydrophilic patches, reservoirs, and physical barriers to fluid flow. Such a plate may have an array of capture probes deposited on the bottom of multiple wells to form a microplate microarray such as the A² Plate by Beckman Coulter, Inc.

The present invention is also directed to a kit for detecting at least one target analyte in a sample, the kit comprising: a solid substrate for containing a sample; at least one primary capture probe bound to the solid substrate, the primary capture probe being bindable to a target analyte for forming a primary capture probe/target complex; a secondary capture probe bindable to the primary capture probe/target complex; a reagent bindable to the secondary capture probe to form a label enzyme; and a chemiluminescent reagent that can interact with the label enzyme to form a fluorescent precipitate and at least one of chemiluminescence and a visible precipitate.

The kit is used by adding a sample to the solid substrate at conditions such that a target analyte in the sample binds to the primary capture probe bound to the solid substrate to form a primary capture probe/target complex. The secondary capture probe is added to the solid substrate at conditions such that the secondary capture probe binds to the primary capture probe/target complex.

The reagent bindable to the secondary capture probe is added to form a label enzyme. The chemiluminescent reagent is added to form a fluorescent precipitate. Target analyte in the sample is detected by detecting the presence of any fluorescent precipitate.

### BRIEF DESCRIPTION OF THE DRAWINGS

These features, aspects and advantages of the present invention will become better understood with reference to the following description, appended claims and accompanying figures where:

FIG. 1 is a flowchart of a method of detecting an analyte according to an embodiment of the present invention;

FIG. 2 illustrates a sandwich assay employing a detection reaction according to an embodiment of the present invention; and

FIG. 3 is a series of photomicrographs of an oligonucleotide hybridization assay according to the present invention showing visible, chemiluminescent and fluorescent signals.

### DETAILED DESCRIPTION

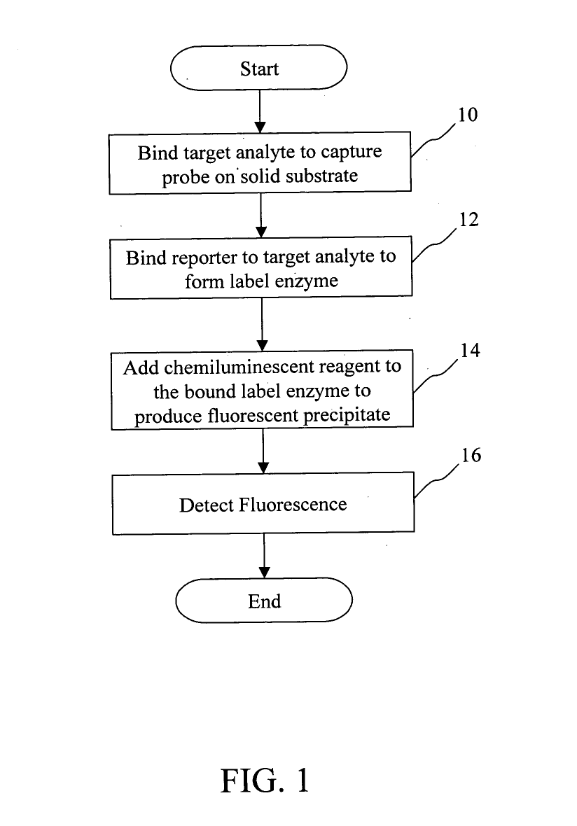

An overview of a method of detecting a target analyte according to an embodiment of the present invention is shown in FIG. 1. The target analyte is bound to a capture probe on a solid substrate, box 10. A reporter is bound to the target analyte to form a label enzyme, box 12. A chemiluminescent reagent is then added to generate a fluorescent precipitate, box 14. The fluorescent precipitate is then detected to reveal the presence of the target analyte, box 16.

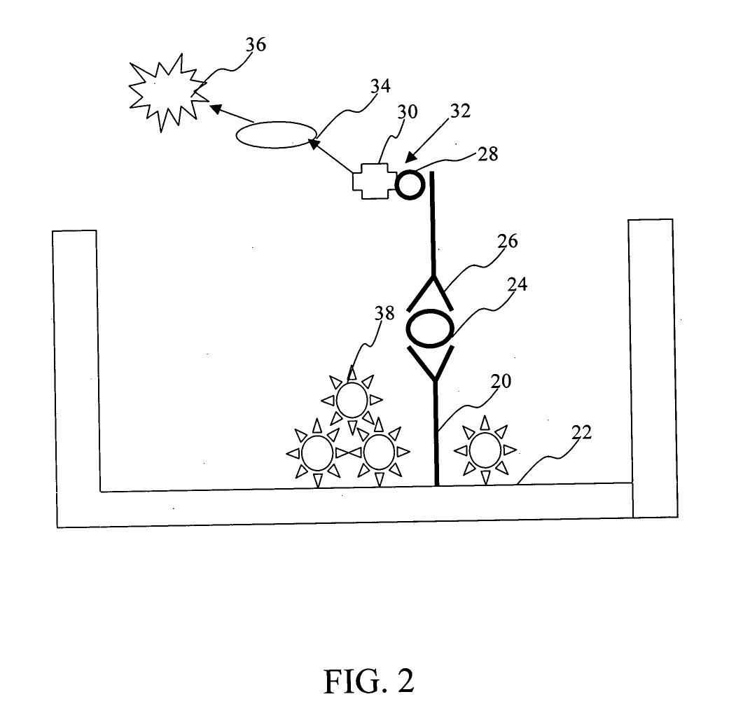

A sandwich assay employing a detection reaction according to an embodiment of the present invention is illustrated in FIG. 2. A primary capture probe 20, such as a primary antibody, is bound to a solid substrate 22. A sample containing a target analyte 24 is mixed with the primary capture probe under conditions suitable for the target analyte to bind to the primary capture probe and form a primary capture probe/target complex bound to the solid substrate.

A secondary capture probe 26, such as a secondary antibody, is mixed with the target analyte and the primary capture probe under conditions suitable for the secondary capture probe to bind to the primary capture probe/target complex. In the present embodiment, the secondary capture probe 26 is biotinylated, and thus, is bound to a plurality of biotin moieties 28.

The primary capture probe/target/secondary capture probe complex is reacted with a conjugate 30. The biotin moieties 28 bind to the conjugate to form a label enzyme 32. The label enzyme then acts on a chemiluminescent reagent 34 to produce chemiluminescence 36 and a fluorescent, chemiluminescent or visible precipitate 38 in the immediate vicinity of the immobilized target analyte 24.

As used herein, “capture probes” generally refer to biomolecules that recognize and bind to target analytes in a sample. Capture probes also recognize and bind to controls.

As used herein, “sample” generally refers to a substance that is being assayed for the presence of one or more target analytes. For example, a sample may be taken using methods known in the art from a cell source or bodily fluid. Non-limiting examples of cell sources available in clinical practice include blood cells, buccal cells, cervicovaginal cells, epithelial cells from urine, fetal cells, or cells present in tissue obtained by biopsy. Body fluids can include blood, urine, cerebrospinal fluid, semen, tissue exudates, saliva, urine and fecal materials.

The primary capture probes, secondary capture probes, and the target analytes may be biomolecules such as nucleic acids, polynucleotides, proteins (i.e., an amino acid sequence containing more than 50 amino acids), peptides (i.e., an amino acid sequence comprising fewer than 50 amino acids); cells and cellular components such as membrane receptors; biomolecule recognition sites, suborganelles, and other structural features. Of particular importance are polypeptides, including aptamers, antibodies, antigens, and enzymes; small compounds such as haptens and peptides; carbohydrates and lipids.

Polynucleotides may be DNA, cDNA, RNA, or a DNA analog, such as PNA (peptide nucleic acid) and LNA (locked nucleic acid). The DNA may be a single- or double-stranded DNA, or a DNA amplified by PCR technique. The RNA may be an mRNA. The length of the polynucleotides may be from about 20 bp to about 10 kb. When the target is a polynucleotide, the primary capture probe (and if present, the secondary capture probe) may comprise a polynucleotide that is complementary to the target polynucleotide or a portion thereof.

Typically, the primary capture probe (and if present, the secondary capture probe) is a monoclonal or polyclonal antibody to the target analyte or a portion thereof, such as a Fab=fragment, that specifically binds to the target analyte. However, as one skilled in the art can readily appreciate, the formation of a specific conjugate comparable to the binding of an antibody to an antigen may be achieved through the use of another specific protein- or peptide-based binding system, such as a receptor protein or fragment thereof and a ligand therefore, which would not generally be considered to involve an immunochemical conjugation. Further, analogs and variants or mimics of various immunoreactants, such as those generated using recombinant DNA techniques, which specifically bind to the target analyte, are contemplated to be within the scope of the present invention.

The Substrate

The substrate may be made of a variety of materials including glasses, thin organic films and polymers. In a preferred embodiment, the substrate is made of polypropylene, polyethylene, and/or their copolymer blends, although other materials and combinations thereof such as polymeric foams, gels, membranes, tapes, glasses or ceramics can be used. Polypropylene is organic material that can be surface activated, but otherwise is chemically inert under harsh chemical conditions. Polypropylene can be used in very corrosive environments. For example, polypropylene has good chemical resistance to a variety of mineral acids (e.g., hydrochloric acid), organic acids (e.g., formic acid, acetic acid), bases (e.g., ammonium hydroxide, potassium hydroxide), salts (e.g., sodium chloride), oxidizing agents (e.g., peracetic acid, iodine solutions), and organic solvents (e.g. acetone, ethyl alcohol, acetonitrile, dichloromethane, etc.). Additionally, polypropylene is hydrophobic and provides a low fluorescence background.

In order to accommodate different testing techniques including specialized testing equipment, the substrate may be molded into a variety of shapes and forms. Examples of shapes and forms of the substrate include, but are not limited to, sheets, particles, microspheres, beads, plates, microplates, compact disks and like structures.

The capture probes are attached to the surface of the substrate by covalent bonding, non-covalent bonding, or any other suitable means, such as affinity interaction with biorecognition molecules attached to the substrate. For example, a covalent attachment using acyl fluoride chemistry may preferably be used. Cells or cellular components may be attached to the substrate via cell surface constituents such as proteins, carbohydrates, glycoproteins or other biomolecules or linkers.

Examples of attachment techniques include chemical cross-linking (U.S. Pat. No. 6,322,968), fixing steps that immobilize adsorbed biomolecules on the substrate (U.S. Pat. No. 4,970,144), the use of adhesive proteins (U.S. Pat. No. 5,024,933) or via direct adsorption on the substrate (U.S. Patent Publication No. 2004/0029156), the entire contents of which are hereby incorporated herein by reference.

Known methods of depositing probes can be used to prepare ordered arrays of probes with each probe located at a site-specific location. For example, thermal inkjet printing techniques utilizing commercially available jet printers and piezoelectric microjet printing techniques, as described in U.S. Pat. No. 4,877,745, the entire contents of which are hereby incorporated by reference, can be utilized to spot selected capture probes on selected substrate surface sites.

The present invention may be a part of a variety of devices, such as microtiter plates, test tubes, inorganic sheets, dipsticks, etc. The particular device is, in and of itself, unimportant, as long as the substrate may be securely affixed to the device without affecting the functional behavior of the substrate, any bound probes, target analytes, and reagents. The device should also be stable to any introduced materials (e.g., clinical samples, etc.).

While the detection system and method of the present invention can be used in conjunction with any suitable solid substrate, the preferred substrate is a multiple array microplate, the subject of U.S. application Ser. No. 09/675,020, the entire contents of which are hereby incorporated by reference. This multiple array microplate is a device comprised of multiple wells, wherein the wells are discrete areas separated by barriers such as walls, hydrophobic patches, hydrophilic patches, troughs, reservoirs, gaskets, pedestals, or the like, that restrict fluid cross-flow between the discrete areas. An array of immobilized capture probes may be formed within each well.

The number of array elements, which are spots of capture probes, present in an array may range from 1 to about 1536 or more, and preferably from about 4 to about 400. The size of the arrays may be the same or different in different wells. The capture probes in each array may be the same or different.

Capture Probe And Target Analyte Hybridization

Contacting capture probes with target analytes (or hybridization) is conducted under conditions that allow the formation of stable complexes between the probes and the target analytes. For example, when target polynucleotides are contacted with probe polynucleotides bound to the substrate, complementary regions on the target and the probe polynucleotides anneal to each other, forming probe/target complexes.

The selection of such conditions is within the level of skill in the art and include those in which a low, substantially zero, percentage of mismatched hybrids form. The precise conditions depend, however, on the desired selectivity and sensitivity of the assay. Such conditions include, but are not limited to, the hybridization temperature, the ionic strength and viscosity of the buffer, and the respective concentrations of the target analyte and capture probes. Hybridization conditions may be initially chosen to correspond to those known to be suitable in standard procedures for hybridization to filters and then optimized for use with the substrates of the present invention. The conditions suitable for the hybridization of one type of target material would appropriately be adjusted for use with other target materials.

For example, in certain embodiments, target polynucleotides are hybridized to probe polynucleotides at temperatures in the range of from about 20° C. to about 70° C., for a period from about 1 hour to about 24 hours, in a suitable hybridization buffer. Suitable hybridization buffers for use in the practice of the present invention generally contain a high concentration of salt. A typical hybridization buffer contains in the range of from about 2× to about 6×SSC and from about 0.01% to about 0.5% SDS at pH 7-8.

Detection Of Bound Target Analyte

Once the probe/target complex is formed, the substrates are washed under conditions suitable to remove substantially all non-specifically bound target analyte or capture probes. Preferably, the washing is carried out at a temperature in the range of from about 20° C. to about 70° C. with a buffer containing from about 0.1× to about 6×SSC and from about 0.01% to about 0.1% SDS. The most preferred stringency wash conditions for polynucleotides presently include a temperature that is the same as hybridization temperature, and a buffer containing 0.2×SSC and 0.01% SDS.

The target analyte is either directly or indirectly labeled with a reporter to facilitate detection. Preferably, the target analyte is either directly or indirectly biotinylated. One skilled in the art will recognize that depending on the target analyte, the target analyte may be biotinylated prior to binding with the capture probe. Alternatively, the capture probe/target analyte is bound to a biotinylated secondary capture probe, such as a secondary antibody that in-turn reacts with the reporter.

The biotin moieties bind to the reporter to form a label enzyme. Preferably, the capture probe/target analyte complex is reacted with a streptavidin-alkaline phosphatase conjugate or with SA-Dextran-AP conjugate, a dextran polymer of streptavidin and alkaline phosphatase available from DakoCytomation to form a label enzyme. Other enzyme conjugates may be used, such as Amdex-AP available from Beckman Coulter, streptavidin-HRP; or an antibody, polypeptide or nucleic acid conjugate of alkaline phosphatase. Typically, the label enzyme comprises phosphatases or peroxidases such as horseradish peroxidase.

The label enzyme then acts on a chemiluminescent reagent, such as LumiPhos WB available from Pierce Chemical Co., APS-5 available from Lumigen, Inc., and DuoLux available from Vector Labs to produce chemiluminescence. Chemiluminescence at particular locations on the substrate having known, predetermined probes, indicates that certain target analytes have been found. The amount of chemiluminescence can be used to quantify the amount of target analyte found.

Additionally, the cleavage of the chemiluminescent reagent leads to formation of a fluorescent precipitate in the immediate vicinity of the immobilized target analyte. The fluorescent precipitate generated by cleavage of the chemiluminescent reagent is advantageous because it has a high intensity, as the label enzyme causes the breakdown of a large quantity of chemiluminescent reagent over discrete areas. Additionally, the fluorescent precipitate has advantageous excitation and emission wavelengths that eliminate the need for a UV excitation light source and allow for the precipitate to be better distinguished from background fluorescence. The fluorescent signal is long lasting and can be read days or weeks later if properly stored in the dark under refrigeration.

The fluorescence from the fluorescence precipitate may be detected by a naked eye or by means of specially designed instrumentation, such as a confocal array reader. For example, in one embodiment, a fluorescent signal is recorded with a charge coupled device (CCD) camera. Fluorescent signal at particular locations on the substrate having known, predetermined probes, indicates that certain target analytes are present. The strength of the fluorescent signal detected may also be used to quantify the amount of target analyte present.

Preferably, the fluorescent precipitate generated from the chemiluminescent reagent has an excitation wavelength of from about 460 to about 500 nm. In an embodiment, the excitation light used is 480 nm with a 40 nm bandpass filter.

Preferably, the fluorescent precipitate has an emission wavelength from about 500 nm to about 600 nm on polypropylene plastic. In an embodiment the emission light is detected at 535 nm with a 50 nm bandpass filter.

Additionally, the cleavage of the chemiluminescent reagent leads to formation of a visible precipitate in the immediate vicinity of the immobilized target analyte. The visible precipitate is advantageous, because it can be observed with the naked eye, imaged by a CCD camera system, or scanned using a flatbed document scanner.

Quantification of samples may be achieved using one or more of any generated chemiluminescence, fluorescent precipitate or visible precipitate. Preferably, a CCD camera system or similar device captures light output from any chemiluminescent, visible precipitate or fluorescent precipitate. The intensity of the light output is transformed into a convenient unit of measure, such as pixel units. Image analysis software, such as ImaGene from BioDiscovery, Inc., is then used to determine signal and background levels from the captured image files, thereby providing a means to quantify the target analyte from a dose-response curve.

In an additional embodiment of the present invention, the pH of the reaction mixture at the time of addition of the chemiluminescent reagent is increased to allow for increased cleavage of the chemiluminescent reagent. Increased cleavage of the chemiluminescent reagent leads to increased chemiluminescence and more fluorescent precipitate. Preferably the pH of the reaction mixture at the time of addition of the chemiluminescent reagent is increased to at least about pH 9.5.

### EXAMPLE 1

A fluorescence immunoassay was performed using a Beckman Coulter A² MicroArray Cytokine Assay. The assay kit used was a Human Cytokine “4-plex” Assay Kit (Beckman Coulter Part Number A11876).

In the experiment, 200 μL of A² wash buffer (Beckman Coulter Part Number A11877) was added to each well of an A² MicroArray 96-well plate (Beckman Coulter Part Number 140888). The plate was incubated at ambient temperature with shaking for 5 minutes.

Following incubation, 55 μL of a mixture of reference biotinylated oligonucleotide and anticytokine-oligonucleotide conjugates (Beckman Coulter Part Numbers A12802; A14358; A15359; A15360 for IL-1β; IL-4; IL-10; IL-12p70, respectively) was added to the wells. The plate was incubated at ambient temperature with shaking for 1 hour. Following incubation, the mixture was removed and the plate washed 3 times with 200 μL of wash buffer.

Following washing, 70 μL of standard cytokine analytes (Beckman Coulter Part Number A12803) freshly prepared at 1× concentration, then 1:3 serial dilutions in FBS (fetal bovine serum) buffer (Beckman Coulter Part Number A11878) was added to each well. The plate was then incubated at ambient temperature with shaking for 1 hour. Following incubation, the mixture was removed and the plate washed 3 times with 200 μL of wash buffer.

Following washing, 55 μL of the biotinylated anti-cytokine antibody mixture (Beckman Coulter Part Numbers A12804; A15361; A15362; A15363) was added to the wells. The plate was then incubated at ambient temperature with shaking for 1 hour. Following incubation, the mixture was removed and the plate washed 3 times with 200 μL of wash buffer.

Following washing, 70 μL of streptavidin-alkaline phosphatase conjugate (Prozyme Cat. No. CJ30A, 0.1 μg/mL, in diluent) was added to the wells. The plate was then incubated at ambient temperature with shaking for 1 hour. Following incubation, the mixture was removed.

The wells were then washed 3 times with 200 μL, 0.1 M Tris, at pH 9.5. Following washing, 70 μL of Lumi-Phos WB (Cat. No. 34150, Pierce Chemical) was added to each of the wells. Chemiluminescence was produced at this point. The plate was then incubated at ambient temperature with shaking for 1 hour. Following incubation, all of the liquid was removed and the wells read in an A² plate reader (FITC filter cube) at a 250 ms scan rate per well. Both visible and fluorescent precipitate was produced.

### EXAMPLE 2

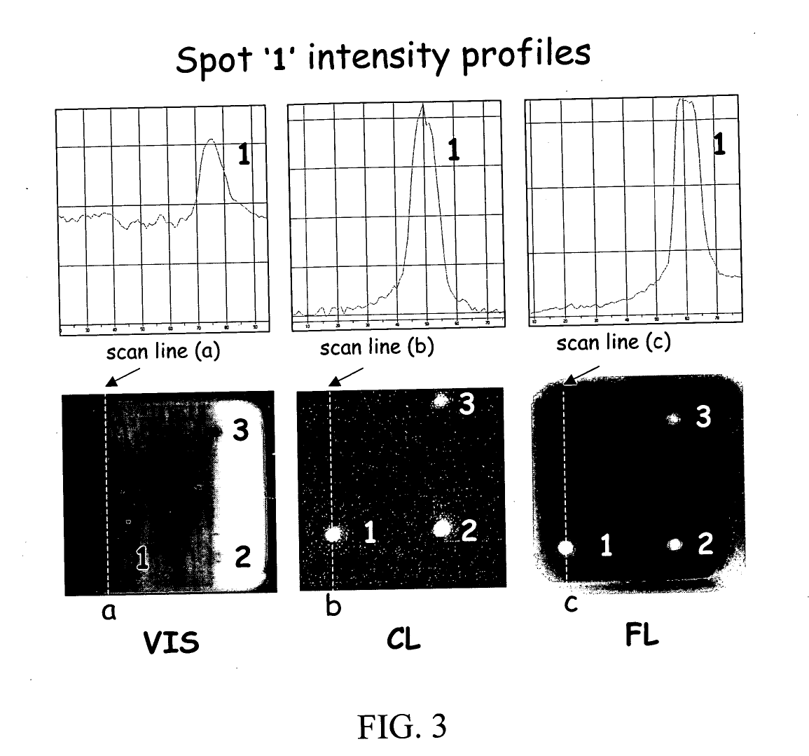

As shown in FIG. 3, a capture probe (30 mer oligonucleotide) was deposited at x,y coordinates in the bottom of a microplate well corresponding to spots 1, 2 & 3. A complementary biotinylated target oligonucleotide was hybridized and the hybrid signal detected using streptavidin-alkaline phosphatase and a chemiluminescent reagent (Lumi Phos WB, Pierce Chemical). A visible (VIS) precipitate, a chemiluminescent (CL) signal and a fluorescent (FL) precipitate were obtained.

As shown in FIG. 3, when the microplate is scanned, signals are obtained from each different product. The scan of the visible precipitate (VIS) is shown in scan line (a). The scan of the chemiluminescent signal (CL) is shown in scan line (b). Additionally, the scan of the fluorescent precipitate (FL) is shown in scan line (c). The signal intensities can reveal the presence of a target analyte. Optionally, the signal intensities can be used to quantify the amount of target analyte present.

Kits

Kits comprising reagents useful for performing the methods of the invention are also provided. In one embodiment, a kit comprises a solid substrate for containing a sample, the solid substrate having at least one capture probe, such as a primary antibody, bound thereto. The kit also comprises a secondary antibody bindable to any primary antibody/target complex following addition of the sample. Also included is a reagent bindable to the secondary antibody to form a label enzyme, and a chemiluminescent reagent that can interact with the label enzyme to form a fluorescent precipitate.

In an embodiment, the reagent bindable to the secondary antibody is at least one of the group consisting of: streptavidin-alkaline phosphatase conjugate and streptavidin-Dextran-alkaline phosphatase conjugate. The chemiluminescent reagent is at least one of the group consisting of LumiPhos WB, APS-5, and DuoLux. The substrate is a multiple array microplate with different primary antibodies bound thereto. In an additional embodiment, the kit includes a detector for detecting fluorescence from the fluorescent precipitate.

The present invention allows for the generation of an advantageous fluorescent precipitate. The fluorescent precipitate generates a very bright fluorescent signal that is much more long lived than the chemiluminescent signal. The fluorescent precipitate formed by the present invention is brighter than existing methods and compositions for generating fluorescent precipitates.

Additionally, the present invention teaches a composition and method for generating a visible precipitate that is viewable with the naked eye either with or without a magnifying apparatus. The visible precipitate allows a viewer to ascertain the presence or absence of target analytes without the need for additional processing, thereby lowering the cost and time needed for analysis.

Having thus described the invention, it should be apparent that numerous modifications and adaptations may be resorted to without departing from the scope and fair meaning of the instant invention as set forth hereinabove and as described hereinbelow by the claims.

Although the present invention has been described in considerable detail with reference to certain preferred versions thereof, other versions are possible. Therefore, the spirit and scope of the appended claims should not be limited to the description of the preferred versions described herein.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function, should not be interpreted as a “means” or “step” clause as specified in 35 U.S.C. §112.

What is claimed is:

1. A method for detecting a target analyte in a sample comprising the steps of:
a. binding the target analyte to a solid substrate;
b. binding a reporter to the bound target to form a label enzyme;
c. contacting a chemiluminescent reagent with the label enzyme to produce a fluorescent precipitate; and
d. detecting fluorescence from the fluorescent precipitate.

2. The method of claim 1 further comprising quantifying the bound target present from the fluorescence detected from the fluorescent precipitate.

3. The method of claim 1 wherein the contacting step further produces chemiluminescence.

4. The method of claim 3 further comprising:
detecting the chemiluminescence produced; and
quantifying the amount of bound target from the detected chemiluminescence.

5. The method of claim 3 wherein the contacting step further produces a visible precipitate.

6. The method of claim 5 further comprising:
detecting the visible precipitate produced; and
quantifying the amount of target bound from the visible precipitate detected.

7. The method of claim 1 wherein the contacting step further produces a visible precipitate.

8. The method of claim 7 further comprising:
detecting the visible precipitate produced; and
quantifying the amount of target bound from the visible precipitate detected.

9. The method of claim 1 wherein the solid substrate comprises at least one of the group consisting of a glass, organic thin film, inorganic thin film, polymer, self-assembled monolayers, and a microparticle.

10. The method of claim 1 wherein the solid substrate is selected from the group consisting of foams, filaments, threads, sheets, films, slides, gels, membranes, tapes, and beads, compact disks and microplates.

11. The method of claim 1 wherein the solid substrate comprises a plate having discrete isolated areas in the form of at least one of the group consisting of wells, troughs, pedestals, hydrophobic patches, hydrophilic patches, reservoirs, and physical barriers to fluid flow.

12. The method of claim 1 wherein the label enzyme comprises at least one of the group consisting of streptavidin-alkaline phosphatase and streptavidin-Dextran-alkaline phosphatase.

13. The method of claim 1 wherein the label enzyme comprises one of the group consisting of phosphatases and peroxidases.

14. The method of claim 13 wherein the label enzyme comprises horseradish peroxidase.

15. The method of claim 1 wherein the fluorescent precipitate has an excitation wavelength of from about 460 nm to about 500 nm.

16. The method of claim 15 wherein the fluorescent precipitate has an emission range of from about 500 nm to about 600 nm.

17. The method of claim 1 wherein the step of binding the target analyte to the solid substrate further comprises:
a. binding a primary capture probe to the solid substrate; and
b. binding the target analyte to the primary capture probe.

18. The method of claim 17 wherein the step of binding a label enzyme to the bound target comprises:
a. binding a biotinylated secondary capture probe to the target analyte; and
b. reacting the biotinylated secondary capture probe with at least of the group consisting of: streptavidin-alkaline phosphatase and streptavidin-Dextran-alkaline phosphatase.

19. The method of claim 18 wherein the step of contacting a chemiluminescent reagent with the bound label enzyme comprises increasing the pH to from at least about 7.5 to about 9.5.

20. A kit for detecting at least one target analyte in a sample, the kit comprising:
a. a solid substrate for containing a sample;
b. at least one primary capture probe bound to the solid substrate, the primary capture probe being bindable to a target analyte for forming a primary capture probe/target complex;
c. a secondary capture probe bindable to the primary capture probe/target complex;
d. a reagent bindable to the secondary capture probe to form a label enzyme; and
e. a chemiluminescent reagent that can interact with the label enzyme to form a fluorescent precipitate.

21. The kit of claim 20 wherein the reagent bindable to the secondary capture probe is at least one of the group consisting of: streptavidin-alkaline phosphatase and streptavidin-Dextran-alkaline phosphatase.

22. The kit of claim 20 further comprising a detector for detecting fluorescence from the fluorescent precipitate.

23. The kit of claim 20 wherein the substrate is a multiple array microplate; and wherein a plurality of different primary capture probes are bound to the substrate.

24. A method of detecting an analyte comprising the steps of:
a. obtaining the kit of claim 20;
b. adding a sample to the solid substrate at conditions such that a target analyte in the sample binds to the primary capture probe bound to the solid substrate to form a primary capture probe/target complex;
c. adding the secondary capture probe to the solid substrate at conditions such that the secondary capture probe binds to the primary capture probe/target complex;
d. mixing the reagent bindable to the secondary capture probe to form a label enzyme;
e. adding the chemiluminescent reagent mixable with the label enzyme to form a fluorescent precipitate; and
f. detecting any target analyte in the sample by detecting the presence of any fluorescent precipitate.
